(19) United States
(12) Patent Application Publication
(10) Pub. No.: US 2011/0195987 A1
(43) Pub. Date: Aug. 11, 2011

(54) TREATMENT WITH CHOLINERGIC AGONISTS

(76) Inventors: Thomas NAJARIAN, Los Osos, CA (US); Kathyrn Susan UNKEL, Los Osos, CA (US)

(21) Appl. No.: 12/898,241

(22) Filed: Oct. 5, 2010

Related U.S. Application Data
(60) Provisional application No. 61/248,739, filed on Oct. 5, 2009.
(60) Provisional application No. 61/299,104, filed on Jan. 28, 2010.

(51) Int. Cl. A61K 31/439; A61K 31/4178; A61K 31/557; A61P 15/00; A61P 35/00; A61P 13/02

(52) U.S. Cl. 514/278; 514/397; 514/573

(57) ABSTRACT

Methods of treating disorders with cholinergic agonists for example, muscarinic receptor agonists such as pilocarpine and cevimeline are provided. In particular, methods of treating and/or preventing interstitial cystitis, yeast infections, urinary tract infections, atrophic vaginitis, vaginal dryness, and sexual dysfunction associated with vaginal dryness by administering a cholinergic agonist to the subject suffering from the disorders are provided. In addition, intra-vaginal administration of cholinergic agonists such as muscarinic receptor agonists to patients suffering from interstitial cystitis, vaginal dryness, and sexual dysfunction associated with vaginal dryness is also provided.

### RELATED APPLICATIONS

This application claims priority to U.S. Provisional Application No. 61/248,739, Attorney Docket No. TNI-012-1, filed Oct. 5, 2009, and U.S. Provisional Application No. 61/299,104, Attorney Docket No. TNI-012-2, filed Jan. 28, 2010. The contents of any patents, patent applications, and references cited throughout this specification are hereby incorporated by reference in their entireties.

### FIELD OF THE INVENTION

The present invention generally relates to methods of treating vaginal disorders with cholinergic agonists. In particular, the present invention relates to methods of treating and/or preventing interstitial cystitis, yeast infections, urinary tract infections, atrophic vaginitis, vaginal dryness, and sexual dysfunction associated with vaginal dryness by administering a cholinergic agonist to the subject suffering from the disorders.

### BACKGROUND

Interstitial cystitis (IC) is a disorder of the bladder, accompanied by urinary urgency and pain in the abdominal region and perineal region, but not accompanied by infections and particular pathologic findings. IC causes very serious symptoms including inflammation of the entire bladder wall extending not only to the mucous membrane but also to the muscle layer. IC is generally a pervasive inflammatory condition of the bladder and can be disabling to a sufferer. Interstitial cystitis is a condition in which very few of the available therapies are effective.

Nearly half of all women between the ages of 40 and 59 suffer from vaginal dryness at some point. For perimenopausal and postmenopausal women, vaginal dryness is often acute, and ranks as one of the top ten problems afflicting postmenopausal women. Vaginal dryness often explains female sexual dysfunction because a lack of vaginal lubrication can make intercourse painful. Women who suffer from vaginal dryness often dread intercourse and avoid sex as a result. Therefore, many women suffering vaginal dryness experience a drop-off in libido.

Accordingly, there remains a need for treatments of vaginal conditions, such as IC, yeast infections, atrophic vaginitis, urinary tract infections, vaginal dryness and/or sexual dysfunction.

### SUMMARY OF THE INVENTION

The present invention is directed to methods and compositions for the treatment and/or prevention of vaginal conditions, such as IC, yeast infections, atrophic vaginitis, urinary tract infections, vaginal dryness and/or sexual dysfunction. The present invention is based, at least in part, on the discovery that vaginal conditions, such as IC, yeast infections, atrophic vaginitis, urinary tract infections, vaginal dryness or sexual dysfunction associated with vaginal dryness, can be effectively treated or prevented by administering an effective amount of a cholinergic agonist to a subject suffering from such disorders.

In one aspect, the invention provides a method of treating or preventing interstitial cystitis by administering to a subject suffering from the disorder, an effective amount of a cholinergic agonist. In another aspect, the invention provides a method of treating or preventing vaginal dryness by administering to a subject suffering from the disorder, an effective amount of a cholinergic agonist. In another aspect, the invention provides a method of treating or preventing sexual dysfunction for example, associated with vaginal dryness, by administering to a subject suffering from the disorder an effective amount of a cholinergic agonist. In another aspect, the invention provides a method of treating or preventing yeast infection by administering to a subject suffering from a yeast infection, an effective amount of cholinergic agonist. In another aspect, the invention provides a method of treating or preventing urinary tract infection by administering to a subject suffering from a urinary tract infection, an effective amount of cholinergic agonist. In another aspect, the invention provides a method of treating or preventing atrophic vaginitis by administering to a subject suffering from atrophic vaginitis, an effective amount of cholinergic agonist. In yet another aspect, the invention provides a method of reducing, eliminating or avoiding side effects, for example: nausea, sweating or excessive heart rate resulting from treatment with a cholinergic agonist by administering the cholinergic agonist to a subject intra-vaginally.

In certain embodiments of each of the foregoing aspects of the invention, the invention provides methods where the cholinergic agonist is a muscarinic receptor agonist. For example, the muscarinic receptor agonist can be selected from the group consisting of pilocarpine, cevimeline, sabcomeline, xenomaline, aceclidine, arecoline, muscarine, or any combination thereof. In various embodiments, the invention provides methods where the muscarinic receptor agonist is pilocarpine or cevimeline.

In some embodiments, about 0.2 to about 200 mg of pilocarpine or cevimeline is administered. In certain embodiments, about 5 to about 15 mg of pilocarpine is administered. In various embodiments, about 10 to about 60 mg of cevimeline is administered.

In various embodiments, the invention provides methods where the cholinergic agonist, for example, a muscarinic receptor agonist, is administered intra-vaginally. In some embodiments, the invention provides methods where the cholinergic agonist is administered topically or via mucous membrane. In certain embodiments, the invention provides methods where the cholinergic agonist is administered as a suppository.

In various embodiments, the cholinergic agonist can be administered before, after or concurrently with a female sex hormone, a lubricant, a spermicidal agent, a contraceptive agent, or any combination thereof.

For example, the female sex hormone can be selected from the group consisting of a prostaglandin, prostaglandin agonist, estrogen or estrogen analog, progesterone or progesterone analog, estradiol, medroxyprogesterone and testosterone or testosterone analog. In a particular embodiment, the invention provides methods where the female sex hormone is a prostaglandin or prostaglandin agonist. In various embodiments, the invention provides methods where the female sex hormone is alprostadil. In some embodiments, the invention provides methods where about 20 to about 2000 mcg, about 100 to about 1000 mcg, or about 200 to about 800 mcg of alprostadil is administered.

In some embodiments, the invention provides methods where the cholinergic agonist is administered before, after or concurrently with hormone replacement therapy.

In various embodiments, the invention provides methods where the subject has or has had cancer. In some embodiments, the invention provides methods where the cancer is breast cancer. In certain embodiments, the invention provides methods where the cholinergic agent is administered for example, intra-vaginally, when hormone replacement therapy is contraindicated for example, where the subject has breast cancer.

In another aspect, the invention provides a pharmaceutical composition including a cholinergic agonist in an amount effective to treat or prevent vaginal dryness, sexual dysfunction, atrophic vaginitis, yeast infections, urinary tract infections or interstitial cystitis, and a pharmaceutically acceptable carrier or diluent. In some embodiments, the cholinergic agonist is a muscarinic receptor agonist.

In certain embodiments, the invention provides pharmaceutical compositions where the composition is suitable for intra-vaginal administration. In various embodiments, the invention provides pharmaceutical compositions where the composition is suitable for administration topically or via mucous membrane. In certain embodiments, the invention provides pharmaceutical compositions including about 0.2 to about 200 mg of the cholinergic agonist. In various embodiments, the invention provides pharmaceutical compositions including about 2 to about 60 mg of the cholinergic agonist.

In some embodiments, the pharmaceutical compositions can further include a female sex hormone, a lubricant, a spermicidal agent, a contraceptive agent, or any combination thereof. For example, the female sex hormone can be selected from the group consisting of a prostaglandin, prostaglandin agonist, estrogen or estrogen agonist, progesterone or progesterone agonist and testosterone or testosterone agonist. In various embodiments, the invention provides pharmaceutical compositions where the female sex hormone is a prostaglandin or prostaglandin agonist. In some embodiments, the invention provides pharmaceutical compositions where the female sex hormone is alprostadil.

In some embodiments, the invention provides pharmaceutical compositions including about 20 to about 2000 mcg of the female sex hormone. In certain embodiments, the invention provides pharmaceutical compositions including about 100 to about 1000 mcg of the female sex hormone. In various embodiments, the invention provides pharmaceutical compositions including about 200 to about 800 mcg of the female sex hormone.

In another aspect, the invention provides a vaginal suppository including the pharmaceutical formulation as described above.

In yet another aspect, the invention provides a kit including the pharmaceutical composition as described above and instructions for using the pharmaceutical composition for treating or preventing interstitial cystitis, atrophic vaginitis, yeast infections, urinary tract infections, vaginal dryness, or sexual dysfunction.

The foregoing brief summary broadly describes the features and technical advantages of certain embodiments of the present invention. Further technical advantages can be described in the detailed description of the invention that follows. Novel features that are believed to be characteristic of the invention can be better understood from the detailed description of the invention when considered in connection with any accompanying figures and examples. However, the figures and examples provided herein are intended to help illustrate the invention or assist with developing an understanding of the invention, and are not intended to be definitions of the invention's scope.

### DETAILED DESCRIPTION OF THE INVENTION

The present invention is directed to methods and compositions for the treatment or prevention of vaginal conditions, such as IC, yeast infections, urinary tract infections, atrophic vaginitis, vaginal dryness and sexual dysfunction. The present invention is based, at least in part, on the surprising discovery that cholinergic agonists such as muscarinic receptor agonists including, for example, pilocarpine and cevimeline, can be used to treat vaginal conditions, such as IC, yeast infections, urinary tract infections, atrophic vaginitis, vaginal dryness and sexual dysfunction. Pilocarpine has been previously used as an agent for the treatment of dry mouth associated with Sjogren's syndrome. However, the foregoing use of pilocarpine is very different from the current invention. Indeed, the pathophysiology of Sjogren's syndrome is distinct from that of vaginal conditions such as IC, yeast infections, urinary tract infections, atrophic vaginitis, vaginal dryness and sexual dysfunction.

Moreover, the use of cholinergic agonists, such as, muscarinic receptor agonists, has been found to be surprisingly effective in treating these vaginal conditions. In particular, pilocarpine and cevimeline have been found to be particularly and surprisingly effective in achieving therapeutic effect. In addition, intra-vaginal administration of the cholinergic agonists such as muscarinic receptor agonists has been found to be surprisingly effective in reducing and/or relieving side effects such as nausea, sweating and elevated heart rates associated with, for example, oral treatment with cholinergic agonists and, in particular, muscarinic receptor agonists.

Accordingly, the present invention provides compositions for the treatment or prevention of vaginal conditions, such as IC, yeast infections, urinary tract infections, atrophic vaginitis, vaginal dryness and/or sexual dysfunction and methods for such treatment or prevention by administering cholinergic agonists such as muscarinic receptor agonists.

As used herein, the term “Interstitial Cystitis” or “IC” refers to the art recognized urinary bladder disease characterized by inflammation and/or pain in the bladder and associated with urination (dysuria), urinary frequency (as often as every 10 minutes), urgency, and pressure in the bladder and/or pelvis.

As used herein, the term “urinary tract infection” refers to the art recognized bacterial infection of the urinary tract. Urinary tract infections are generally caused by invasion of ascending bacteria through the lower urinary tracts. Urinary tract infections can be caused by a variety of bacteria, however, E. coli is the most prevalant cause.

As used herein, the term “yeast infection” refers to the art recognized fungal infection of the vagina, most commonly due to the fungus Candida albicans. As used herein, the term “atrophic vaginitis” refers to the art recognized atrophy of the vagina resulting from a decrease in estrogen production, for example, during menopause. Atrophic vaginitis is commonly associated with dryness, soreness, irritation, and dyspareunia and renders the vaginal epithelium more susceptible to infection and secondary inflammation.

The term “vaginal dryness” includes loss of or lack of natural lubrication of the vagina. Natural lubrication in the vagina is produced from secretion by the glands such as Bartholin's glands generally present in the vaginal canal. Vaginal dryness as used herein includes insufficient secretion of the natural lubricating fluid for example, caused by stress, emotional disorders, change in hormonal levels and side effects to certain therapies. Poor lubrication can result from insufficient excitement and stimulation, or from hormonal changes caused by menopause, pregnancy, breast-feeding, or age. Irritation from contraceptive creams and foams can also cause vaginal dryness, as can fear and anxiety about sex.

The term “sexual dysfuction” includes difficulty experienced by an individual or a couple during any stage of a normal sexual activity, including desire, arousal or orgasm. As used herein “sexual dysfunction” includes sexual pain disorders that primarily affect women such as for example, dyspareunia (painful intercourse) or vaginismus (an involuntary spasm of the muscles of the vaginal wall that interferes with intercourse). In a particular embodiment, sexual dysfunction includes difficulty caused by insufficient lubrication (vaginal dryness) in women.

The terms “treat,” “treated,” “treating” or “treatment” include the diminishment or alleviation of at least one symptom associated or caused by the state, disorder or disease being treated. In certain embodiments, the treatment includes the preventing of the induction of a disorder, via the activation of the cholinergic agonist of the invention, which would in turn diminish or alleviate at least one symptom associated or caused by the disorder being treated. For example, treatment can be diminishment of one or several symptoms of a disorder or complete eradication of a disorder.

The terms “prevent,” “prevented,” “preventing,” “prevention” or “prophylaxis” include the prevention or removal of recurrence of at least one symptom associated or caused by the state, disorder or disease being treated. In some embodiments, for example, prevention can include a complete eradication and/or stopping recurrence of one or several symptoms of a disorder.

The term “side effect” includes effects which are undesirable or which act against the intended beneficial effect of an agent of the invention. The term “side effect” encompasses adverse toxic or adverse psychological effects. In certain embodiments, side effects refer to nausea, excessive heart rate and sweating associated with administration, for example, oral administration, of cholinergic agonists.

The term “subject” includes warm-blooded animals, preferably mammals, including humans. In a preferred embodiment, the subject is a primate. In an even more preferred embodiment, the subject is a human.

The terms “administer,” “administered,” or “administering” to a subject include dispensing, delivering or applying a composition including a cholinergic agonist to a subject by any suitable route for delivery of the cholinergic agonist to the desired location in the subject, including delivery by topical application. Alternatively or in combination, delivery is by the parenteral or oral route, buccal administration, transdermal delivery and administration by the rectal, colonic, vaginal, intranasal or respiratory tract route. Preferably, the compositions of the invention are administered topically, e.g., to the skin or mucus membranes of an affected subject.

The term “effective amount” includes an amount effective, at dosages and for periods of time necessary, to achieve the desired result, e.g., sufficient to treat a subject suffering from IC, yeast infections, urinary tract infections, atrophic vaginitis, vaginal dryness and/or sexual dysfunction associated with vaginal dryness; sufficient to prevent IC, yeast infections, urinary tract infections, atrophic vaginitis, vaginal dryness and/or sexual dysfunction associated with vaginal dryness, for example, in a subject likely to develop IC, yeast infections, urinary tract infections, atrophic vaginitis, vaginal dryness and/or sexual dysfunction associated with vaginal dryness; sufficient to treat or alleviate or sufficient to prevent IC, yeast infections, urinary tract infections, atrophic vaginitis, vaginal dryness and/or sexual dysfunction associated with vaginal dryness, for example, in a subject predisposed to such conditions. An effective amount of cholinergic agonist, as defined herein may vary according to factors such as the state, severity and extent of the condition, i.e., IC, yeast infections, urinary tract infections, atrophic vaginitis, vaginal dryness and/or sexual dysfunction associated with vaginal dryness, age, and weight of the subject, and the ability of the cholinergic agonist to elicit a desired response in the subject. Dosage regimens may be adjusted to provide the optimum therapeutic response. An effective amount is also one in which any toxic or detrimental effects (e.g., side effects) of the cholinergic agonist are outweighed by the therapeutically beneficial effects. Specific effective amounts of cholinergic agonists encompassed by the present invention are described below.

The term “cholinergic agonist” includes an agent that interacts with a cholinergic receptor. The interaction may result in an increase in activity of the receptor (i.e., up-regulation). The interaction between a cholinergic agonist of the invention and the cholinergic receptor may be a direct interaction or an indirect interaction. In certain embodiments, the cholinergic agonist is a muscarinic receptor agonist.

The term “muscarinic receptor agonist” includes an agent that interacts with a muscarinic receptor. The interaction may result in an increase in activity of the receptor (i.e., up-regulation). The interaction between a muscarinic receptor agonist of the invention and the muscarinic receptor may be a direct interaction or an indirect interaction. Exemplary muscarinic receptor agonists include, but are not limited to, pilocarpine, cevimeline, sabcomeline, xenomaline, aceclidine, arecoline, muscarine, derivatives thereof or any combination thereof. As used herein pilocarpine refers to (3S,4R)- 3-ethyl- 4-((1-methyl- 1H-imidazol- 5-yl) methyl)dihydrofuran- 2(3H)-one. As used herein cevimeline refers to 2-methyspiro (1,3-oxathiolane-5,3) quinuclidine.

The term “intra-vaginal” includes a means of delivery of an agent of the invention into the vaginal cavity of a mammal such that the agent can be absorbed systemically by the mammal therefrom so as to achieve or suppress some physiological effect.

The term “topical” includes administration of an agent of the invention to the skin surface or mucous membrane of a subject, including humans, so that the agent passes through the skin tissue. The term “mucous membrane” includes the moist linings of the buccal cavity, nasal cavity, gastrointestinal tract, respiratory tract, conjunctiva, vagina, colon, urinary bladder, and urethra. Accordingly, in a particular embodiment, the term “topical” encompasses administration to the vaginal membrane.

The term “suppository” includes a unit dose of an agent of the invention having a homogeneous solid form at room temperature which melts rapidly in human ducts, as well as a “rectal capsule” of a unit dose of soft capsule filled with the dispersed substance. The soft capsule is designed to disintegrate in human coelomic ducts to release an agent of the invention contained therein in a time dependent manner. In various embodiments, the term “suppository” encompasses a vaginal suppository.

Compositions Comprising Cholinergic Agonists

The present invention is directed, in part, to compositions comprising cholinergic agonists, a pharmaceutically acceptable salt thereof or a combination of compounds which allow for the release of cholinergic agonist in vivo. The cholinergic agonist is present in the compositions in an amount effective to treat, for example, alleviate, or prevent IC. Alternatively, the cholinergic agonist is present in the compositions in an amount sufficient to treat, for example, alleviate, or prevent yeast infections, urinary tract infections, atrophic vaginitis, vaginal dryness and/or sexual dysfunction associated with vaginal dryness.

A variety of cholinergic agonists, for example, muscarine, pilocarpine, cevimeline, nicotine, suxamethonium, physostigmine, and neostigmine can be used in the present invention. A variety of cholinergic agonists are commercially available in pharmaceutical formulations, such as for example, acetylcholine, bethanechol, carbachol, methacholine, arecoline, nicotine, muscarine, pilocarpine, cevimeline, donepezil, edrophonium, neostigmine, physostigmine, pyridostigmine, rivastigmine, tacrine, echothiophate, isoflurophate, malathion, cisapride, metoclopramide, clonidine, propranolol, atenolol, prazosin, and methyldopa. In some embodiments, commercially available cholinergic agonists can be used in the invention directly without further chemical modicications. In certain embodiments, the commercially available cholinergic agents can be modified to a pharmaceutical composition that is suitable for the present invention. For example, tablets of commercially available cholinergic agents can be modified to deliver the drug as a topical formulation or as a suppository.

In another aspect, the invention includes administering a muscarinic receptor agonist. A variety of muscarinic receptor agonists, for example, muscarine, pilocarpine, cevimeline, acetylcholine, oxotremorine, carbachol, McNA343, methacholine, and bethanechol can be used in the present invention. A variety of muscarinic receptor agonists are commercially available, such as for example, pilocarpine, cevimeline, sabcomeline, xenomaline, aceclidine, arecoline, and muscarine. In some embodiments, commercially available muscarinic receptor agonists can be used in the invention directly without further chemical modicications. In certain embodiments, the commercially available muscarinic receptor agonists can be modified to a pharmaceutical composition that is suitable for the present invention. For example, tablets of commercially available muscarinic receptor agonists can be modified to deliver the drug as a topical formulation or as a suppository.

In some embodiments, an effective amount of a cholinergic agonist, for example muscarinic receptor agonist of the invention is the amount sufficient to treat or prevent a disorder in a subject. The effective amount can vary depending on such factors as the size and weight of the subject, the type of illness, or the particular cholinergic agonist of the invention. For example, the choice of the cholinergic agonist of the invention can affect what constitutes an effective amount. One of ordinary skill in the art would be able to study the factors contained herein and make the determination regarding the effective amount of the cholinergic agonists of the invention without undue experimentation.

The regimen of administration can affect what constitutes an effective amount. A cholinergic agonist of the invention can be administered to the subject either prior to or after the onset of a disorder. Further, several divided dosages, as well as staggered dosages can be administered daily or sequentially, or the dose can be continuously infused. Further, the dosages of the cholinergic agonist(s) of the invention can be proportionally increased or decreased as indicated by the exigencies of the therapeutic or prophylactic situation.

The cholinergic agonist or muscarinic receptor agonist may be present in the compositions at a concentration of about 0.1% to about 100% w/w. Alternatively, the cholinergic agonist may be present in the compositions at a concentration selected from the group consisting of about 1% to about 100% w/w, about 2% to about 75% w/w, about 3% to about 50% w/w and about 4% to about 25% w/w. The cholinergic agonist may be present at about at least 1%, 2%, 3%, 4%, 5%, 6%, 7%, 8%, 9%, 10% or 15% w/w. Ranges intermediate to the above recited amounts, e.g., about 2% to about 90% w/w, are also intended to be part of this invention. For example, ranges of values using a combination of any of the above recited values as upper and/or lower limits are intended to be included.

The concentration of cholinergic agonist may depend on the nature of the composition. For example, when the formulation is designed as a cream, the formulation may include about 1 to about 10% of cholinergic agonist. Alternatively, when the formulation is designed as a suppository, the formulation may include about 5 to about 100% of cholinergic agonist.

In some embodiments, the initial pharmaceutically effective amount of the cholinergic agonist administered per dose can be in the range of about 0.001-100 mg/kg, namely about 0.01 to 20 mg/kg of patient body weight per day. In some embodiments, a single dose of the cholinergic agonist administered to a subject is about 0.2 to about 200 mg. In certain embodiments, a single dose of the cholinergic agonist administered to a subject is about 0.5 to about 100 mg. In various embodiments, a single dose of the cholinergic agonist administered to a subject is about 2 to about 60 mg. In certain embodiments, at least 0.001 mg, 0.01 mg, 0.1 mg, 1.0 mg, 2 mg, 3 mg, 4 mg, 5 mg, 10 mg, 15 mg, 20 mg, 25 mg, 30 mg, 35 mg, 40 mg, 45 mg, 50 mg, 55 mg, and 60 mg of the cholinergic agonist can be administered. In some embodiments, a single dose of the cholinergic agonist for example, pilocarpine, administered to a subject is about 5 to about 15 mg. In certain embodiments, a single does of the cholinergic agonist for example, cevimeline, administered to a subject is about 10 to about 60 mg.

The cholinergic agonist compositions of the present invention can be the only active ingredient(s) formulated into the pharmaceutical composition, although in certain embodiments the cholinergic agonist may be combined with one or more other active ingredients including, but not limited to, a female sex hormone, a lubricant, a spermicidal agent, a contraceptive agent, or any combination thereof.

In some embodiments, the initial pharmaceutically effective amount of the female sex hormone administered per dose can be in the range of about 0.001-100 mcg/kg, namely about 0.01 to 20 mcg/kg of patient body weight per day. In some embodiments, a single dose of the female sex hormone administered to a subject is about 20 to about 2000 mcg. In certain embodiments, a single dose of the female sex hormone administered to a subject is about 100 to about 1000 mcg. In various embodiments, a single dose of the female sex hormone administered to a subject is about 200 to about 800 mcg. In certain embodiments, at least 0.001 mcg, 0.01 mcg, 0.1 mcg, 1.0 mcg, 2 mcg, 3 mcg, 4 mcg, 5 mcg, 10 mcg, 15 mcg, 20 mcg, 25 mcg, 50 mcg, 75 mcg, 100 mcg, 150 mcg, 200 mcg, 300 mcg, 400 mcg, 500 mcg, 600 mcg, 700 mcg, or 800 mcg of the female sex hormone can be administered. In some embodiments, about 0.5 mg to about 10 mg of the female sex hormone for example, estradiol can be administered. In certain embodiments, about 1 mg to about 3 mg of the female sex hormone for example, estradiol can be administered. In various embodiments, about 1 mg to about 200 mg of the female sex hormone for example, progesterone, dose range 50-200 mg, or medroxyprogesterone, dose range 1-10mg can be administered for example intra-vaginally. In some embodiments, about 2 mg to about 100 mg of the female sex hormone for example, progesterone or medroxyprogesterone can be administered. In various embodiments, about 0.1 mg to about 50 mg of the female sex hormone for example, testosterone can be administered for example intra-vaginally. In some embodiments, about 1 mg to about 5 mg of the female sex hormone for example, testosterone can be administered.

In each of the above embodiments, the amount of female sex hormone can be in the range of about 0.5% w/w to about 50% w/w. In some embodiments, the amount of female sex hormone can be in the range of about 1% w/w to about 25% w/w. In certain embodiments, the amount of female sex hormone can be in the range of about 2% w/w to about 5% w/w.

In one embodiment, the cholinergic agonist is co-administered to a subject before, after or concurrently with a female sex hormone, a lubricant, a spermicidal agent, a contraceptive agent, or any combination thereof.

Varieties of lubricants, for example, water-based, oil-based, silicone-based and organic lubricants, can be used directly with or without further modifications.

Varieties of spermicidal agents, for example, octoxynol-9, nonoxynol-9 and neem extract can be used directly with or without further modifications.

Varieties of contraceptive agents, for example, ormeloxifene, medroxyprogesterone acetate, and Norethindrone acetate can be used directly with or without further modifications.

In a particular embodiment, the cholinergic agonist composition is suitable for topical administration. For example, the composition may be in the form of a gel, liquid or spray to allow, for example, for topical application to areas associated with IC. Alternatively, the composition may be in the form of a solution, lotion, mask, soap, moisturizer, powder, perfume, dye, brilliantine, aerosol, pomade, cream, ointment or paste.

When formulated in an ointment, the active cholinergic agonist can be employed with either a paraffinic or a water-miscible ointment base. Alternatively, the active cholinergic agonist can be formulated in a cream with an oil-in-water cream base.

If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a cholinergic agonist which enhances absorption or penetration of the active cholinergic agonist through the skin or other affected areas. Examples of such dermal penetration enhancers include dimethyl sulfoxide and related analogs.

In another embodiment, the cholinergic agonist is administered to the subject using a pharmaceutical composition comprising a solid ionic complex of the cholinergic agonist and a carrier macromolecule, where the cholinergic agonist content of the complex is at least 0.05% by weight, preferably at least 0.05%, 0.10%, 0.15%, 0.20%, 0.25%, 0.30%, 0.35%, 0.40%, 0.45%, 0.50%, 0.55%, 0.60%, 0.65%, 0.70%, 0.75%, 0.80%, 0.85%, 0.90%, 0.95%, 1.0%, 2.0%, 3.0%, 4.0%, 5.0%, 7.0%, 10.0%, 12.0% or 15.0% w/w. Ranges intermediate to the above recited amounts, e.g., about 0.08% to about 13.0% w/w, are also intended to be part of this invention. For example, ranges of values using a combination of any of the above recited values as upper and/or lower limits are intended to be included.

Alternatively or in combination, the cholinergic agonist may be present as a pharmaceutically acceptable salt. Moreover, alternatively or in combination, the composition may include chemical compounds which react or combine to form and release in vivo cholinergic agonist.

The compositions of the present invention may further include other agents, for example, inactive carriers. Inactive carriers including, but not limited to, deionized water, arachidyl alcohol, behenyl alcohol, arachidyl glucoside, montanoV 202, cetearyl alcohol, capric caprilic triglyceride, isopropyl palmitate, steareth-2, dimethicon, steareth-20, allantoin, propylene glycol, Methylisothiazolinone (sheromix MT), sodium benzoate, and combinations thereof may also be included. Moreover, the compositions of the invention may further include additional pharmaceutically and/or cosmetically acceptable compounds and/or compositions.

In one embodiment, the pharmaceutical composition comprises cholinergic agonist and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. Preferably, the carrier is suitable for topical administration, for example, to the skin. Alternatively, the carrier can be suitable for intra-vaginal administration. In another embodiment, the carrier is suitable for oral administration. Pharmaceutically acceptable carriers include sterile aqueous solutions or dispersions and sterile powders for the extemporaneous preparation of sterile solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

A typical formulation can be prepared by mixing a cholinergic agonist of the present invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used can depend upon the means and purpose for which the cholinergic agonist of the present invention is being applied. Solvents are generally selected based on solvents recognized by persons skilled in the art as safe to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a cholinergic agonist of the present invention or pharmaceutical composition thereof) or aid in the manufacturing of the pharmaceutical product (i.e., medicament).

The formulations can be prepared using conventional dissolution and mixing procedures. For example, the bulk drug substance (i.e., cholinergic agonist of the present invention or stabilized form of the cholinergic agonist, such as a complex with a cyclodextrin derivative or other known complexation agent) is dissolved in a suitable solvent in the presence of one or more of the excipients described above. The cholinergic agonist of the present invention is typically formulated into pharmaceutical dosage forms to provide an easily controllable dosage of the drug and to enable patient compliance with the prescribed regimen.

The pharmaceutical compositions of the invention can be formulated, dosed, and administered in a fashion, i.e. amounts, concentrations, schedules, course, vehicles, and route of administration, consistent with good medical practice. Factors for consideration in this context include the particular disorder being treated, the particular mammal being treated, the clinical condition of the individual patient, the cause of the disorder, the site of delivery of the cholinergic agonist, the method of administration, the scheduling of administration, and other factors known to medical practitioners. The “therapeutically effective amount” of the cholinergic agonist to be administered can be governed by such considerations, and is the minimum amount necessary to prevent, ameliorate, or treat the disorders such as IC, vaginal dryness or sexual dysfunction associated with vaginal dryness. Such amount is preferably below the amount that is toxic to the host or renders the host significantly more susceptible to side effects.

The formulations include those suitable for the administration routes detailed herein. The formulations may conveniently be presented in unit dosage form and can be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations can be prepared by uniformly and intimately bringing into association the active ingredient with liquid carriers or finely divided solid carriers or both, and then, if necessary, shaping the product.

Formulations of the cholinergic agonists suitable for administration can be prepared as discrete units such as pills, capsules, cachets or tablets each containing a predetermined amount of the cholinergic agonists.

Tablets, troches, aqueous or oil suspensions, dispersible powders or granules, emulsions, hard or soft capsules, e.g. gelatin capsules, syrups or elixirs can be prepared. Formulations of a cholinergic agonist can be prepared according to any method known to the art for the manufacture of pharmaceutical compositions and such compositions may contain one or more cholinergic agonists including coloring agents and preserving agents. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and absorption and thereby provide a sustained action over a longer period. For example, a time delay material such as glyceryl monostearate or glyceryl distearate alone or with a wax can be employed.

Aqueous suspensions of the invention contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxy-benzoate, and one or more coloring agents.

The pharmaceutical composition of a cholinergic agonist, for example muscarinic receptor agonists can be in the form of a sterile preparation, such as a sterile aqueous or oleaginous suspension. This suspension can be formulated according to the known art using those suitable dispersing or wetting agents and suspending agents which have been mentioned above. The sterile preparation can also be a sterile solution or suspension in a non-toxic acceptable diluent or solvent, such as a solution in 1,3-butane-diol or prepared as a powder. Among the acceptable vehicles and solvents that can be employed are water, Ringer's solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation.

Therapeutic compositions typically can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it can be preferable to include isotonic agents, for example, sugars, polyalcohols such as mannitol, sorbitol, or sodium chloride in the composition. Prolonged absorption of the compositions can be brought about by including in the composition an agent which delays absorption, for example, monostearate salts and gelatin.

The oily phase of the emulsions of this invention can be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both, an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The cholinergic agonist compositions can be formulated with one or more additional compounds that enhance the solubility of the cholinergic agonist. Preferred compounds to be added to formulations to enhance the solubility of the cholinergic agonist are cyclodextrin derivatives, preferably hydroxypropyl-y-cyclodextrin. For example, inclusion in the formulation of hydroxypropyl-y-cyclodextrin at a concentration 50-200 mM may increase the aqueous solubility of the cholinergic agonist.

Another formulation for the cholinergic agonist comprises the detergent Tween-80, polyethylene glycol (PEG) and ethanol in a saline solution. A non-limiting example of such a preferred formulation is 0.16% Tween-80, 1.3% PEG-3000 and 2% ethanol in saline.

In another embodiment, a pharmaceutical formulation of the invention is a sterile formulation. For example, following formation of the water-insoluble complex, the complex can be sterilized, preferably by gamma irradiation or electron beam sterilization. Alternatively, to prepare a sterile pharmaceutical formulation, the water-insoluble complex can be isolated using conventional sterile techniques (e.g., using sterile starting materials and carrying out the production process aseptically).

In certain embodiments, the compounds of the invention can be administered in a time release or a sustained release formulation, for example in a composition which includes a slow release polymer. The cholinergic agonist compositions can be prepared with carriers that can protect the cholinergic agonist against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

As used herein, the term “time release” or “sustained release” is intended to refer to continual delivery of cholinergic agonist for example, muscarinic receptor agonists in vivo over a period of time following administration, preferably at least several days, a week or several weeks and up to a month or more. In a preferred embodiment, a formulation of the invention achieves sustained delivery for at least about 7, 14, 21 or 28 days, at which point the sustained release formulation can be re-administered to achieve sustained delivery for another 28 day period (which re-administration can be repeated every 7, 14, 21 or 28 days to achieve sustained delivery for several months to years). Sustained delivery of the cholinergic agonist can be demonstrated by, for example, the continued therapeutic effect of the cholinergic agonist over time. Alternatively, sustained delivery of the cholinergic agonist may be demonstrated by detecting the presence of the cholinergic agonist in vivo over time.

Suitable examples of sustained-release preparations include semipermeable matrices of solid hydrophobic polymers containing the cholinergic agonist(s), which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (microspheres composed of lactic acid-glycolic acid copolymer and leuprolide acetate), and poly-D-(-)-3-hydroxybutyric acid.

Advantageously, the present invention also provides kits for use by a consumer for treating disease. The invention also provides kits for use by health providers, hospital staff and technicians. For example, the invention provides kits for using the cholinergic agonists for example, muscarinic receptor agonists of the invention for treatment or prevention of IC, vaginal dryness, or disorders associated with vaginal dryness such as sexual dysfunction. The kits can comprise a) a pharmaceutical composition comprising a cholinergic agonist for example, muscarinic receptor agonists and a pharmaceutically acceptable carrier, vehicle or diluent; and, optionally, b) instructions describing a method of using the pharmaceutical composition for treating the specific disorder. The instructions may also indicate that the kit is for treating disorder while substantially reducing the concomitant liability of adverse effects associated with administration of the composition. In certain embodiments, the kits can comprise (i) a cholinergic agonist for example, muscarinic receptor agonists, with instructions for mixing or combining with (ii) another cholinergic agonist, female sex hormone, lubricant, spermicidal agent, or a contraceptive agent.

A “kit” as used in the instant application can include a container for containing the separate unit dosage forms such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle which is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time. Preferably, the dispenser is equipped with a memory-aid, so as to further facilitate compliance with the regimen. An example of such a memory-aid is a mechanical counter, which indicates the number of daily doses that, has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Methods of Treatment and Prevention of IC

The present invention is further directed, in part, to methods of treatment and/ or prevention of IC by administering to a subject an effective amount of a composition comprising cholinergic agonist for example, muscarinic receptor agonists, a pharmaceutically acceptable salt thereof, or a combination of compounds which allow for the release of cholinergic agonist in vivo, thereby treating and/ or preventing IC.

The compositions of the present invention may be administered as necessary to achieve the desired effect and depend on a variety of factors including, but not limited to, the severity of the condition, age and history of the subject and the nature of the composition, for example, concentration of cholinergic agonist and/or sustained release capabilities. In various embodiments, the compositions may be administered at least two, three, four, five or six times a day. Additionally, the therapeutic or preventative regimens may cover a period of at least 2, 3, 4, 5, 6, 7, 8, 9, 10, 11, 12, 13, 14, 15, 16, 17, 18, 19, 20, 21, 22, 23 or 24 weeks.

The amount of active cholinergic agonist that can be combined with the carrier material to produce a single dosage form can vary depending upon the host treated and the particular mode of administration. For example, a time-release formulation intended for administration to humans may contain approximately 0.001 to 1000 mg of active cholinergic agonist compounded with an appropriate and convenient amount of carrier material which may vary from about 5 to about 95% of the total compositions (weight:weight). The pharmaceutical composition can be prepared to provide easily measurable amounts for administration. For example, an aqueous solution may contain from about 3 to 500 mug of the active cholinergic agonist per milliliter of solution in order that infusion of a suitable volume at a rate of about 30 mL/hr can occur.

Formulations suitable for administration include aqueous and non-aqueous sterile solutions which may contain anti-oxidants, buffers, bacteriostats and solutes which render the formulation isotonic with the blood of the intended recipient; and aqueous and non-aqueous sterile suspensions which may include suspending agents and thickening agents.

In various embodiments, the methods of the present invention further comprise monitoring the effectiveness of treatment. For example, the amount of dryness, ulceration, redness, infection, pain, urgency, and/or fibroids associated with IC may be monitored.

The effectiveness of the treatments described herein can be assessed based on a variety of factors including, for example, the disappearance of dryness, ulceration, redness, infection, pain, urgency, and/or fibroids; a reduction in size and/or severity of the dryness, ulceration, redness, infection, pain, urgency, and/or fibroids; and/or a reduction of the number of ulcerations, infection spots, pain, urgency, and/or fibroids.

In certain embodiments, the methods of the present invention involve co-administration of cholinergic agonist, a pharmaceutically acceptable salt thereof, or a combination of compounds which release cholinergic agonist in vivo, with an additional active agent including, but not limited to, a female sex hormone, a lubricant, a spermicidal agent, a contraceptive agent, other agents having therapeutic or preventative effect against IC and/or any combinations thereof.

In certain embodiments, the female sex hormone can be selected from a prostaglandin, prostaglandin agonist, estrogen, progesterone, testosterone, estradiol, medroxyprogesterone, or any combination thereof. In some embodiments, the female sex hormone can be a prostaglandin or prostaglandin agonist. In various preferred embodiments, the female sex hormone can be alprostadil. In certain embodiments, the cholinergic agonist can be administered before, after or concurrently with a female sex hormone, a lubricant, a spermicidal agent, a contraceptive agent, or any combination thereof.

Subjects suitable for treatment using the regimens of the present invention should have or are susceptible to developing IC. IC can be diagnosed through routine examinations, for example, through use of a bright light or magnifying lens. Additionally, blood, urine, and other bodily fluids can be examined to confirm the diagnosis.

In addition, subjects susceptible and predisposed to developing IC can be identified by consideration of a variety of factors. For example, older subjects who are at least 30, 35, 40, 45, 50, 55, 60, 65 and 70 years old are susceptible to the development of IC are suitable candidates for the methods of the present invention. In certain embodiments, women in the peri-menopausal or post-menopausal stage can be suitable candidates. In addition, subjects who are immunocompromised can be particularly prone to developing IC.

Accordingly, in one aspect, the invention is directed to a method of treating or preventing IC in a subject by selecting a subject who is susceptible to the development of IC and administering to the subject an effective amount of a composition comprising cholinergic agonist, a pharmaceutically acceptable salt thereof, or a combination of compounds to allow for the release of cholinergic agonist in vivo, thereby treating or preventing IC in the subject. As set forth above, in various embodiments, the subject may be older than about 30, 35, 40, 45, 50, 55, 60, 65 and 70 years old.

Methods of Treatment and Prevention of Yeast Infections, Urinary Tract Infections and Atrophic Vaginitis

The present invention is further directed, in part, to methods of treatment and/ or prevention of at least one of yeast infections, urinary tract infections and atrophic vaginitis by administering to a subject an effective amount of a composition comprising cholinergic agonist, for example, muscarinic receptor agonists, a pharmaceutically acceptable salt thereof, or a combination of compounds which allow for the release of cholinergic agonist in vivo, thereby treating and/ or preventing the disorder(s).

The compositions of the present invention may be administered as necessary to achieve the desired effect and depend on a variety of factors including, but not limited to, the severity of the condition, age and history of the subject and the nature of the composition, for example, concentration of cholinergic agonist and/or sustained release capabilities. In various embodiments, the compositions may be administered at least two, three, four, five or six times a day. Additionally, the therapeutic or preventative regimens may cover a period of at least 2, 3, 4, 5, 6, 7, 8, 9, 10, 11, 12, 13, 14, 15, 16, 17, 18, 19, 20, 21, 22, 23 or 24 weeks.

The amount of active cholinergic agonist that can be combined with the carrier material to produce a single dosage form can vary depending upon the host treated and the particular mode of administration. For example, a time-release formulation intended for administration to humans may contain approximately 0.001 to 1000 mg of active cholinergic agonist compounded with an appropriate and convenient amount of carrier material which may vary from about 5 to about 95% of the total compositions (weight:weight). The pharmaceutical composition can be prepared to provide easily measurable amounts for administration. For example, an aqueous solution may contain from about 3 to 500 mg of the active cholinergic agonist per milliliter of solution in order that infusion of a suitable volume at a rate of about 30 mL/hr can occur.

Formulations suitable for administration include aqueous and non-aqueous sterile solutions which may contain anti-oxidants, buffers, bacteriostats and solutes which render the formulation isotonic with the blood of the intended recipient; and aqueous and non-aqueous sterile suspensions which may include suspending agents and thickening agents.

In various embodiments, the methods of the present invention further comprise monitoring the effectiveness of treatment. For example, frequency of urination, blood and/or pus in urine, cloudiness of urine and/or urinary incontinence may be monitored. Additionally, the amount of dryness, ulceration, redness, infection, pain and/or urgency associated with the condition, i.e., yeast infection, urinary tract infection and/or atrophic vaginitis, may be monitored.

The effectiveness of the treatments described herein can be assessed based on a variety of factors including, for example, the disappearance of dryness, ulceration, redness, infection, pain and/or urgency; a reduction in size and/or severity of the dryness, ulceration, redness, infection, pain and/or urgency; and/or a reduction of the number of ulcerations, infection spots, pain and/or urgency. Alternatively, the disappearance or reduction in frequency of urination, blood and/or pus in urine, cloudiness of urine and/or urinary incontinence may be assessed as indicative of effective treatment.

In certain embodiments, the methods of the present invention involve co-administration of cholinergic agonist, a pharmaceutically acceptable salt thereof, or a combination of compounds which release cholinergic agonist in vivo, with additional active agents having therapeutic or preventative effect against yeast infection, urinary tract infections and/or atrophic vaginitis.

Subjects suitable for treatment using the regimens of the present invention should have or are susceptible to developing yeast infection, urinary tract infections and/or atrophic vaginitis. These conditions can be diagnosed through routine examinations, for example of the urinary tract, or through examination of blood, urine, and other bodily fluids.

Accordingly, in one aspect, the invention is directed to a method of treating or preventing yeast infection, urinary tract infection and/or atrophic vaginitis in a subject by selecting a subject who is susceptible to the development of the disorder and administering to the subject an effective amount of a composition comprising cholinergic agonist, a pharmaceutically acceptable salt thereof, or a combination of compounds to allow for the release of cholinergic agonist in vivo, thereby treating or preventing the disorder in the subject.

Methods of Treatment and Prevention of Vaginal Dryness and/or Sexual Dysfunction Associated with Vaginal Dryness

The present invention is further directed, in part, to methods of treatment and prevention of vaginal dryness and/or sexual dysfunction associated with vaginal dryness by administering to a subject an effective amount of a composition comprising cholinergic agonist such as muscarinic receptor agonists, for example, pilocarpine or cevimeline, a pharmaceutically acceptable salt thereof, or a combination of compounds which allow for the release of cholinergic agonist in vivo, thereby treating or preventing vaginal dryness and/or sexual dysfunction associated with vaginal dryness.

As above with respect to treatment and prevention of IC, the compositions of the present invention may be administered as necessary to achieve the desired effect. Accordingly, the regimen of administration depends on a variety of factors including, but not limited to, the severity of the condition, age and history of the subject and the nature of the composition, for example, concentration of cholinergic agonist and/or sustained release capabilities. In various embodiments, the compositions may be administered at least two, three, four, five or six times a day. Additionally, the therapeutic or preventative regimens may cover a period of at least 2, 3, 4, 5, 6, 7, 8, 9, 10, 11, 12, 13, 14, 15, 16, 17, 18, 19, 20, 21, 22, 23 or 24 weeks.

In various embodiments, the methods of the present invention further comprise monitoring the effectiveness of treatment. For example, the amount of vaginal dryness, redness, sexual discomfort, and/or pain associated with vaginal dryness and/or sexual dysfunction associated with vaginal dryness may be monitored.

The effectiveness of the treatments described herein can be assessed based on a variety of factors including, for example, the disappearance of vaginal dryness, redness, sexual discomfort, and/or pain; a reduction in size and/or severity of the vaginal dryness, redness, sexual discomfort, and/or pain.

In certain embodiments, the methods of the present invention involve co-administration of cholinergic agonist, a pharmaceutically acceptable salt thereof, or a combination of compounds which release cholinergic agonist in vivo, with an additional active agent including, but not limited to, a female sex hormone, a lubricant, a spermicidal agent, a contraceptive agent, other agents having therapeutic or preventative effect against vaginal dryness and/or sexual dysfunction associated with vaginal dryness and/or any combinations thereof.

Subjects suitable for treatment using the regimens of the present invention should have or are susceptible to developing vaginal dryness and/or sexual dysfunction associated with vaginal dryness. Vaginal dryness and/or sexual dysfunction associated with vaginal dryness can be diagnosed through routine examinations, for example, through use of a bright light or magnifying lens. Additionally, a questionnaire answered by a subject including addressing the amount, frequency and severity of vaginal dryness and/or sexual dysfunction associated with vaginal dryness can confirm the diagnosis.

In addition, subjects susceptible and predisposed to developing vaginal dryness and/or sexual dysfunction associated with vaginal dryness can be identified by consideration of a variety of factors. For example, older female subjects who are at least 30, 35, 40, 45, 50, 55, 60, 65 and 70 years old are susceptible to the development of vaginal dryness and/or sexual dysfunction associated with vaginal dryness are suitable candidates for the methods of the present invention. In certain embodiments, women in the peri-menopausal or post-menopausal stage can be suitable candidates. In addition, female subjects who are immunocompromised can be particularly prone to developing vaginal dryness and/or sexual dysfunction associated with vaginal dryness.

Accordingly, in one aspect, the invention is directed to a method of treating or preventing vaginal dryness and/or sexual dysfunction associated with vaginal dryness in a female subject by selecting a female subject who is susceptible to the development of vaginal dryness and/or sexual dysfunction associated with vaginal dryness and administering to the female subject an effective amount of a composition comprising a cholinergic agonist, for example, muscarinic receptor agonists, a pharmaceutically acceptable salt thereof, or a combination of compounds to allow for the release of cholinergic agonist in vivo, thereby treating or preventing vaginal dryness and/or sexual dysfunction associated with vaginal dryness in the female subject. As set forth above, in various embodiments, the female subject may be older than about 30, 35, 40, 45, 50, 55, 60, 65 and 70 years old.

In another aspect, the invention is directed to a method of treating or preventing vaginal dryness and/or sexual dysfunction associated with vaginal dryness in a female subject by selecting a female subject who is susceptible to the development of vaginal dryness and/or sexual dysfunction associated with vaginal dryness and administering to the female subject an effective amount of pilocarpine, or a pharmaceutically acceptable salt thereof.

In another aspect, the invention is directed to a method of treating or preventing vaginal dryness and/or sexual dysfunction associated with vaginal dryness in a female subject by selecting a female subject who is susceptible to the development of vaginal dryness and/or sexual dysfunction associated with vaginal dryness and administering to the female subject an effective amount of cevimeline, or a pharmaceutically acceptable salt thereof.

In another aspect, provided herein is the use of a composition comprising a cholinergic agonist, for example, muscarinic receptor agonists, a pharmaceutically acceptable salt thereof, or a combination of compounds to allow for the release of cholinergic agonist in vivo, for the manufacture of a medicament for treating or preventing vaginal dryness and/or sexual dysfunction associated with vaginal dryness in a female subject.

In another aspect, provided herein is the use of cevimeline, or a pharmaceutically acceptable salt thereof, for the manufacture of a medicament for treating or preventing vaginal dryness and/or sexual dysfunction associated with vaginal dryness in a female subject.

In another aspect, provided herein is the use of pilocarpine, or a pharmaceutically acceptable salt thereof, for the manufacture of a medicament for treating or preventing vaginal dryness and/or sexual dysfunction associated with vaginal dryness in a female subject.

Methods of Reducing, Eliminating or Avoiding Side Effects

The present invention is further directed, in part, to methods of reducing, eliminating or avoiding side effects associated with the treatment and prevention of IC, vaginal dryness and/or sexual dysfunction associated with vaginal dryness by intra vaginally administering to a subject an effective amount of a composition comprising cholinergic agonist such as muscarinic receptor agonists, for example, pilocarpine or cevimeline, a pharmaceutically acceptable salt thereof, or a combination of compounds which allow for the release of cholinergic agonist in vivo, thereby reducing, eliminating or avoiding side effects caused as a result of oral administration.

The compositions of the present invention may be administered intra vaginally as necessary to achieve the desired effect. Accordingly, the regimen of intra vaginal administration depends on a variety of factors including, but not limited to, the severity of the condition, age and history of the subject and the nature of the composition, for example, concentration of cholinergic agonist and/or sustained release capabilities. In various embodiments, the compositions may be administered at least two, three, four, five or six times a day. Additionally, the therapeutic or preventative regimens may cover a period of at least 2, 3, 4, 5, 6, 7, 8, 9, 10, 11, 12, 13, 14, 15, 16, 17, 18, 19, 20, 21, 22, 23 or 24 weeks.

In various embodiments, the methods of the present invention further comprise monitoring the effectiveness of treatment by intra vaginal administration. For example, the amount and severity of nausea, sweating and/or excessive heart rate associated with the treatment of vaginal dryness and/or sexual dysfunction associated with vaginal dryness using cholinergic agonists, for example muscarinic receptor agonists may be monitored.

Side effects, for example, nausea, sweating or excessive heart rate, associated with oral administration of cholinergic agonists can be diagnosed through routine examinations, for example, through use of a bright light or magnifying lens. Additionally, a questionnaire answered by a subject including addressing the amount, frequency and severity of nausea, sweating and/or excessive heart rate associated with the treatment of IC, vaginal dryness and/or sexual dysfunction associated with vaginal dryness using cholinergic agonists can confirm the diagnosis.

In certain embodiments, the cholinergic agonists, for example muscarinic receptor agonists can be administered topically to the skin surface or the vaginal membrane in the vagina of the subject. In some embodiments, the cholinergic agonists, for example muscarinic receptor agonists can be administered as a vaginal suppository into the vaginal canal of the subject.

Accordingly, side effects associated with the oral administration likely caused by metabolism of the cholinergic agonists for example, muscarinic receptor agonists in the gastro-intestinal tract can be reduced, eliminated or avoided while maintaining or enhancing the efficacy of the treatment.

The following examples illustrate the preparation of certain specific cholinergic agonists according to the present technology. A skilled artisan appreciates that the invention is not limited to the exemplary work described or to the specific details set forth in the examples.

### EXAMPLES

Case Study 1

In the case of a female patient, KSU, female, DOB Jan. 5, 1955, who has been diagnosed with IC for more than 8 years, no prior treatment has helped the episodes of pain, urinary frequency, and systemic symptoms mentioned above. Previously, estrogen replacement seemed to alleviate some of the severity of symptoms. However, the onset of breast cancer and its treatment have precluded the use of Hormone Replacement Therapy (HRT). Because of vaginal dryness, a trial of Evoxac (cevimeline HCl) capsules, 30 mg daily, by oral administration was tried. To the patient's surprise, in addition to improvement in vaginal dryness, the symptoms of IC also remitted. This lasted for about 2 months. The Evoxac was started on or about Apr. 5, 2009. The oral administration of Evoxac, however, caused some side effects including nausea and rapid heart rate. Metoprolol at a dose of 12.5-25 mg/day was started to slow the heart rate. Because of side effects, the Evoxac was stopped for a few weeks, only to have the symptoms of IC return within a week of discontinuing the medication. The Evoxac was restarted at a dose of 15 mg daily. A trial of intra-vaginal medication at a dose of 30 mg per vagina (powder was taken out of the capsule and instilled using a gel cap) was also tried. Both the 30 mg intra-vaginal, and the 15 mg oral dose improved the symptoms of IC better than any prior treatment including a trial of Elmiron 100 mg TID for four months. Surprisingly, the intra-vaginal administration of 30 mg of Evoxac completely eliminated side-effects that were observed during the oral administration.

Case Study 2

A second patient, SC, female, DOB Nov. 4, 1948, suffering from painful intercourse and vaginal dryness and discomfort, also used oral pilocarpine at a dose of 2.5-5 mg BID. Her vaginal dryness was improved, however, she suffered from excess sweating and nausea. A trial of intra-vaginal administration of pilocarpine, starting at a dose of 1/4 of a 5 mg tablet daily at night was tried. The dose was gradually increased to 7.5 mg daily. At 2.5 mg there was a partial response of increased vaginal secretions, that improved greatly at the 5 and 7.5 mg doses intra-vaginally. The patient had previously tried HRT without much improvement. The improvement in vaginal symptoms was stunning. The patient and her husband could enjoy intercourse for the first time in many months. There were no systemic symptoms from the intra-vaginal pilocarpine.

Case Study 3

A third patient, a female, DS, Sep. 22, 1957, has had problems with vaginal dryness and painful intercourse for several years. Oral hormone replacement therapy consisting of both estradiol and PROMETRIUM (progesterone), had provided no relief for her vaginal symptoms. She was started on oral Evoxac, 30 mg daily. After a week, she administered 30 mg cevimeline mixed with a small amount of yogurt directly in the vagina nightly. She has reported improvement in both vaginal dryness and pain on intercourse and continues on the treatment.

Case Study 4

A fourth patient, a female, LP, DOB Jul. 30, 1950, suffered from vaginal dryness and had a history of urinary tract infections. She was started on oral pilocarpine, 5 mg twice a day. Within a few days, she noticed marked improvement in her symptoms, i.e., a reduction in vaginal dryness and symptoms associated with urinary tract infections. In order to minimize unnecessary side effects associated with oral administration, the patient began taking the medication intermittently with satisfactory results.

Case Study 5

A fifth patient, a female CW, DOB Jun. 4, 1973, suffered from vaginal dryness, frequent urinary tract infections, and frequent vaginal yeast infections. She began and tolerated oral pilocarpine, 5 mg twice a day. Shortly after starting treatment, she reported no more vaginal dryness and far fewer vaginal yeast infections and urinary tract infections during the approximately 8 months on treatment.

Case Study 6

A sixth patient, a female, TT, DOB Jun. 11, 1953, had suffered from frequent urinary tract infections, vaginal dryness, and painful intercourse for several years. She began a week long regimen of oral pilocarpine, 5 mg twice a day, followed by 10 mg placed directly in the vagina nightly. She reported improvement in symptoms associated with urinary tract infections, vaginal dryness and painful intercourse. She has noted that, at times, the 5 mg pilocarpine tablets do not completely dissolve in the vagina. Formulation of the pilocarpine in a manner suitable for intravaginal administration will improve delivery of the pilocarpine and the efficacy of treatment.

Case Study 7

A seventh patient, a female, RL, DOB Aug. 5, 1973, has suffered with symptoms of interstitial cystitis and mild vaginal dryness for many years. Her symptoms consisted of vaginal and pelvic pain as well as signs of urinary tract infections with trace positive cultures. Prior attempts to treat urinary tract infections with antibiotics often failed. She began a regimen of oral pilocarpine, 5 mg twice a day, for a week followed by 5 mg pilocarpine administered directly to the vagina nightly for a week. Subsequently, the amount of pilocarpine was increased to 10 mg directly administered to the vagina nightly. She reported that after the first week of intravaginal administration, there was an increase in vaginal secretions. Vaginal secretions increased more significantly upon increase of dosage to 10 mg pilocarpine. Additionally, she reported a reduction in pelvic pain and vaginal dryness a few days after commencing intravaginal administration of 10 mg pilocarpine.

The present invention and its embodiments have been described in detail. However, the scope of the present invention is not intended to be limited to the particular embodiments of any process, manufacture, composition of matter, cholinergic agonists, means, methods, and/or steps described in the specification. Various modifications, substitutions, and variations can be made to the disclosed material without departing from the spirit and/or essential characteristics of the present invention. Accordingly, one of ordinary skill in the art can readily appreciate from the disclosure that later modifications, substitutions, and/or variations performing substantially the same function or achieving substantially the same result as embodiments described herein can be utilized according to such related embodiments of the present invention. Thus, the following claims are intended to encompass within their scope modifications, substitutions, and variations to processes, manufactures, compositions of matter, cholinergic agonists, means, methods, and/or steps disclosed herein.

The contents of any patents, patent applications, and references cited throughout the specification are herein incorporated by reference in their entireties.

1-30. (canceled)

31. A pharmaceutical composition comprising
a cholinergic agonist in an amount effective to treat or prevent vaginal dryness, sexual dysfunction, atrophic vaginitis, yeast infection, urinary tract infections or interstitial cystitis, and a pharmaceutically acceptable carrier or diluent.

32. (canceled)

33. The pharmaceutical composition of claim 31, wherein the composition is suitable for intra-vaginal administration.

34-43. (canceled)

44. The pharmaceutical composition of claim 33, comprising a vaginal suppository.

45. A kit comprising the pharmaceutical composition of claim 31 and instructions for using the pharmaceutical composition for treating or preventing interstitial cystitis, yeast infection, urinary tract infection, atrophic vaginitis, vaginal dryness, or sexual dysfunction.

46. A method for treating interstitial cystitis comprising administering an effective amount of a cholinergic agonist to a subject in need of such treatment.

47. The method of claim 46, wherein the cholinergic agonist is administered intravaginally.

48. The method of claim 47, wherein the cholinergic agonist is a muscarinic receptor agonist.

49. The method of claim 48, wherein the muscarinic receptor agonist is selected from pilocarpine, cevimeline, sabcomeline, xenomaline, aceclidine, arecoline, muscarine, pharmaceutically acceptable salts thereof, and combinations of any of the foregoing.

50. The method of claim 49, wherein the muscarinic receptor agonist is pilocarpine or a pharmaceutically acceptable salt thereof.

51. The method of claim 50, wherein the effective amount of pilocarpine is about 0.2 mg to 200 mg.

52. The method of claim 51, wherein the effective amount of pilocarpine is about 0.5 mg to 100 mg.

53. The method of claim 52, wherein the effective amount of pilocarpine is about 2 mg to 60 mg.

54. The method of claim 53, wherein the effective amount of pilocarpine is about 5 mg to 15 mg.

55. The method of claim 49, wherein the muscarinic receptor agonist is cevimeline or a pharmaceutically acceptable salt thereof.

56. The method of claim 55, wherein the effective amount of cevimeline is about 0.2 mg to 200 mg.

57. The method of claim 56, wherein the effective amount of cevimeline is about 10 mg to 60 mg.

58. The method of claim 47, wherein the cholinergic agonist is intravaginally administered as a suppository.

59. The method of claim 46, wherein the cholinergic agonist is administered before, after, or concurrently with an effective amount of an additional agent selected from a female sex hormone, testosterone, a testosterone agonist, a prostaglandin, a prostaglandin agonist, a lubricant, a spermicidal agent, a contraceptive agent, and combinations thereof.

60. The method of claim 59, wherein the additional agent is a female sex hormone.

61. The method of claim 59, wherein the additional agent is a prostaglandin.

62. The method of claim 61, wherein the prostaglandin is alprostadil.

63. The method of claim 62, wherein effective amount of alprostadil is about 20 mcg to about 2000 mcg.

64. The method of claim 63, wherein effective amount of alprostadil is about 100 mcg to about 1000 mcg.

65. The method of claim 64, wherein effective amount of alprostadil is about 200 mcg to about 800 mcg.

66. The method of claim 46, wherein the cholinergic agonist is administered before, after, or concurrently with hormone replacement therapy.

67. The method of claim 46, wherein the subject has or has had cancer.

68. The method of claim 67, wherein the cancer is breast cancer.

69. A method for treating a vaginal condition comprising administering to a subject in need of such treatment an effective amount of a cholinergic agonist.

70. The method of claim 69, wherein the cholinergic agonist is administered intravaginally.

71. The method of claim 69, wherein the vaginal condition comprises vaginal dryness, sexual dysfunction, yeast infection, and/or atrophic vaginitis.

72. A method for treating urinary tract infection comprising administering to a subject in need of such treatment an effective amount of a cholinergic agonist.

73. A method for reducing, eliminating, or avoiding side effects resulting from treatment of a female subject with a cholinergic agonist comprising administering the cholinergic agonist to the subject intravaginally.
